(19) United States
(12) Patent Application Publication
(10) Pub. No.: US 2009/0182355 A1
(43) Pub. Date: Jul. 16, 2009

(54) Porous barbs for long-term anchoring in the gastrointestinal tract

(76) Inventors: Andy H. Levine, Newton, MA (US); David A. Melanson, Hudson, NH (US); Ezra S. Fishman, Cambridge, MA (US); Ronald B. Lamport, Pelham, NH (US); James Loper, Wales, MA (US); John Panek, Peabody, MA (US); Sean K. Holmes, West Roxbury, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/316,264

(22) Filed: Dec. 10, 2008

Related U.S. Application Data
(60) Provisional application No. 61/008,400, filed on Dec. 20, 2007.
(60) Provisional application No. 61/133,312, filed on Jun. 27, 2008.

(51) Int. Cl. A61B 17/08

(52) U.S. Cl. 606/153; 606/151

(57) ABSTRACT

Gastrointestinal implants in areas such as the esophageal area, the stomach, and the intestinal area are used in the treatment of conditions like obesity and diabetes. An implant including an anchor with barbs having pores, can allow for longer term anchoring. The pores can promote tissue ingrowth from the surrounding tissue that the barb is penetrating, thus advantageously allowing increased stability and longer term anchoring compared to a non-porous barb.

### RELATED APPLICATIONS

This application claims the benefit of U.S. Provisional Application No. 61/008,400, filed on Dec. 20, 2007, and also claims the benefit of U.S. Provisional Application No. 61/133,312, filed on Jun. 27, 2008.

The entire teachings of the above applications are incorporated herein by reference.

### BACKGROUND OF THE INVENTION

Gastrointestinal implants in the esophagus, stomach and intestines have been proposed to treat the diseases of obesity and Type-2 diabetes mellitus. These include gastrointestinal liners, duodenal pacers, stomach pacers and stomach volume partitioning and bypass devices. As these diseases are chronic in nature, it is desirable to implant therapeutic devices for long periods of time. However, long-term anchoring of devices in the gastrointestinal tract beyond 6 months is difficult and has not been accomplished. For example, consistent anchoring of a gastrointestinal liner implant in the duodenum for 6 months has been accomplished, but 12 months and beyond has proven difficult.

Barbs have been used in gastrointestinal implants to pierce into the muscle layer of the intestines (duodenum) to hold these implants in place. However, as the tissue heals in response to the barbs' piercing, the barbs disengage from the muscle layer into the less dense inflammatory tissue that surrounds the barbs. This leads to the device becoming unstable and subsequently migrating or otherwise moving from its desired location.

As with any implant, it is also desired to be able to remove the implant if the patient so desires or if the implant stops functioning or becomes problematic.

To meet the long term treatment needs of obese and diabetic patients, an anchoring solution that maintains position for more than 6 months, or preferentially permanently, is desirable. It is also desirable to be able to easily remove the implant from the body.

### SUMMARY

The present invention relates to methods and devices for longer term anchoring of gastrointestinal implants in the gastrointestinal tract of a human. This can be accomplished by anchoring the implant with an anchor having porous barbs to promote tissue ingrowth from the surrounding tissue.

The current device is a gastrointestinal implant that includes an anchor and barbs, the barbs that are attached to the anchor having pores.

In one embodiment, the gastrointestinal implant further comprises a floppy, flexible sleeve that is at least a foot in length. The anchor can be a wave anchor.

In one embodiment, the pore size of the barb is between 100 and 400 microns. The barb pores have an open space density from 25% to 75%. The barb can be from 2.0-4.0 mm in height from an anchor strut. In one embodiment, the barb is a rectangular paddle-shaped barb. The rectangular paddle barb can have a width of 1.0-2.0 mm. Alternatively, the barb can also be a hollow barb with a diameter of 0.5-1.0 mm. The barb can also be solid with sintering on its outside, or it may be fully sintered.

In one embodiment, the barb is a barb substrate with a porous sheath covering at least a portion of the barb. The porous sheath can be made of a biocompatible plastic, such as silicone, urethane or HEMA. An inner diameter of the porous sheath is from about 0.25-1.0 mm. The porous sheath can have a wall thickness of at least 200 microns.

Further described is a method of attaching an implant, including positioning the anchor within a natural bodily lumen, the anchor being coupled to a proximal portion of a gastrointestinal implant device, piercing surrounding tissue with barbs that are coupled to the anchor, the barbs having pores, and allowing tissue to grow into the pores of the barbs. The anchor is at least 50.0 mm in diameter, and can be a wave anchor. The barbs are configured to pierce muscle. The anchor is preferably anchored distal to the pylorus in the intestine.

Also described is a gastrointestinal implant comprising a flexible, floppy sleeve, open at both ends, a self expanding anchor and barbs coupled to the anchor. The barb comprises a barb substrate, wherein at least a portion of the barb is textured.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-1D show prior art barbs attached to an anchor;

FIG. 2 illustrates an embodiment of a rectangular paddle-shaped barb;

FIG. 3 illustrated an embodiment of a hollow barb with pores;

FIG. 4 illustrates an embodiment of a sintered barb;

FIGS. 5A-5B illustrate an embodiment of a barb with a porous sheath;

FIG. 6 illustrates an embodiment of a rigid barb with pores;

FIG. 7 illustrates an example of a gastrointestinal implant; and

FIGS. 8A-8B illustrate an example of a gastrointestinal implant with a restrictive feature.

### DETAILED DESCRIPTION

A description of preferred embodiments of the invention follows.

Gastrointestinal implants can be used for a number of treatments. Implants placed within the gastrointestinal tract are typically subject to substantial mechanical forces related to the digestion process.

At least some anchoring devices for anchoring an implant use an interference fit, placing an implant device having a relaxed diameter larger than the diameter offered by the intestine. Other anchoring devices use barbs that are adapted to penetrate into the surrounding muscular tissue of the gastrointestinal tract. Examples of gastrointestinal implants and anchors used for anchoring implants are described in U.S. Pat. No. 7,025,791 B2, incorporated herein in its entirety by reference.

To date, only short-term anchoring (3-6 months) of gastrointestinal implants has been achievable. A good analogy for the etiology of the loosening of the barb from the surrounding tissue is to think of the barbs as a splinter. When a splinter is driven into soft tissue, like the skin, a foreign body immune response is initiated. The response is intended to prevent infection by isolating the splinter from the inside of the body. The body accomplishes this by encapsulating the splinter with inflammatory cells which seal it off. The isolated splinter can then be slowly pushed backward out of the skin which simultaneously heals along the splinter pathway.

In the case of the intestine, the lumen within the intestines is the outside of the body relative to the intestinal tissue. To prevent infection, the intestines encapsulate the barb, and drive it back into the intestinal lumen where it can be expelled from the body.

To meet the long-term treatment needs of obese and diabetic patients for example, an anchoring solution that maintains its position for more than 12 months, or preferably permanently, is needed.

Within this application is described the use of controlled porosity in barbs of a gastrointestinal anchor to achieve long-term and/or permanent anchoring of devices within the gastrointestinal tract. This can be accomplished by several means, many of which will be described herein.

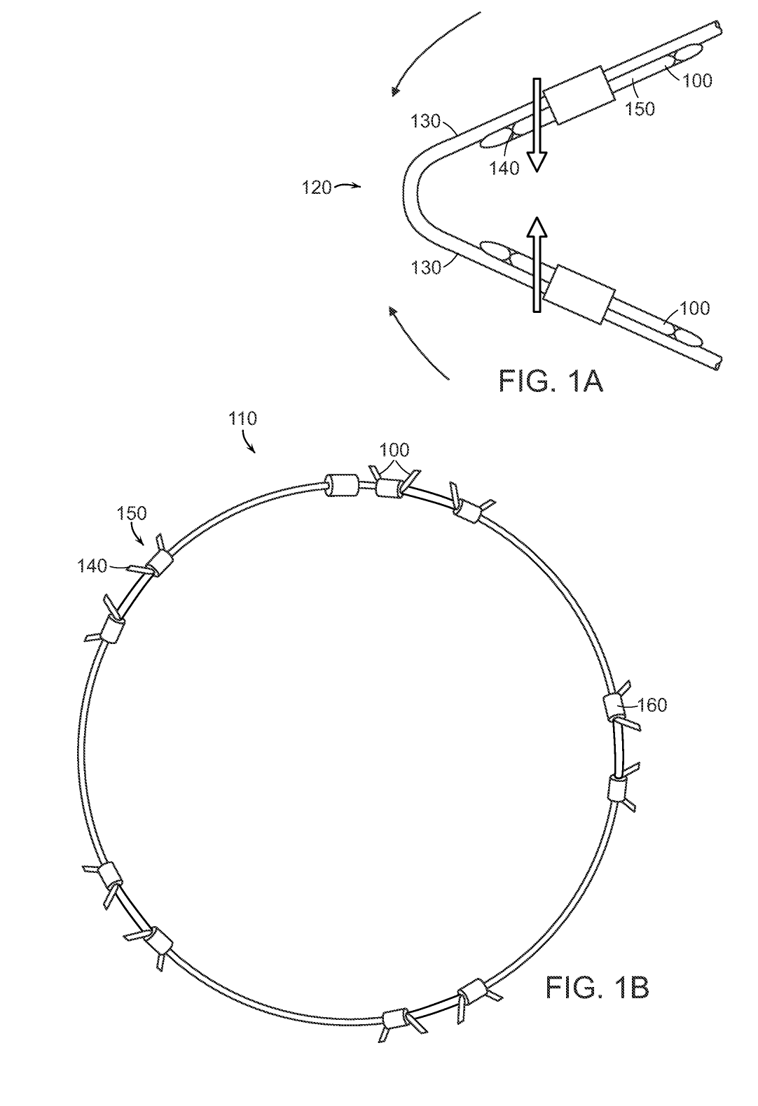

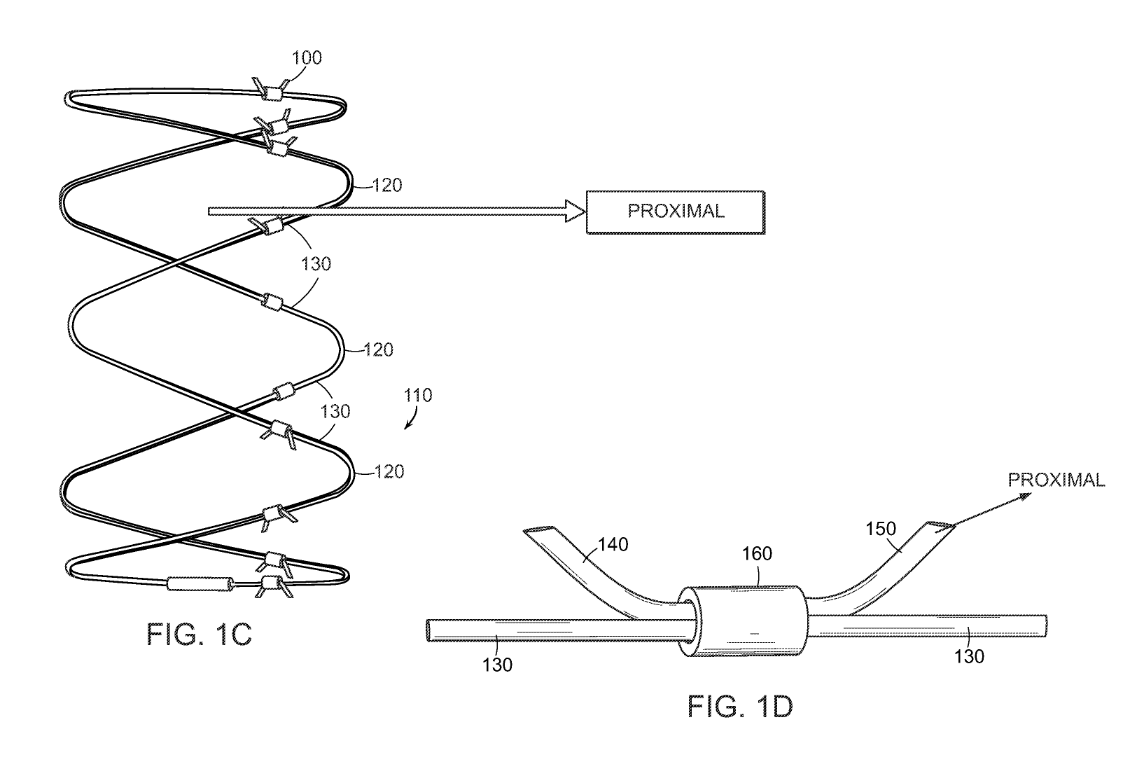

FIGS. 1A-1D show an embodiment of barbs as seen in the prior art that have been used in conjunction with an anchor 110 in the gastrointestinal tract. An end-on view of the anchor 110 with barbs 100 is shown in FIG. 1B. A side view of the anchor 110 with barbs 100 is shown in FIG. 1C.

The anchor 110 may be, for example, a collapsible self-expanding stent with a more complex network of struts. This anchor 110 is a collapsible, self-expanding wave type stent. Alternatively, the wave anchor 110 includes adjacent interconnected struts 130 connected by wave peaks 120 as shown in FIGS. 1A and 1C. In one embodiment, the anchor 110 has ten struts. Wave anchors are described in allowed U.S. patent application Ser. No. 10/858,852 and U.S. patent application Ser. No. 10/858,851, both filed on Jun. 1, 2004, incorporated herein in their entireties by reference.

The anchor 110 includes a plurality of opposed, bi-directional barb pairs 100 for anchoring an implant device within the gastrointestinal tract. The barb pairs 100 include pairs of rigid, elongated barbs. The barbs of the pair, respectively barb 140 and barb 150, are outwardly directed at fixed angles and in opposite axial directions. The barbs are of a length such that they are able to penetrate surrounding tissue. For example, if in the intestine, the barbs should be of a sufficient length to penetrate muscular tissue of the intestinal wall.

Further details of the barbs 100 are shown in FIGS. 1C-1D. Each of struts 130 has one pair of barbs 100 with both proximal and distal facing points. The barb pairs 100 are made from a single piece of wire and are crimped onto each strut 130 with a piece of stainless steel, titanium or nitinol tubing 160. The barb pairs 100 are located at the proximal end of the anchor 110. Each barb 100 is crimped such that the barb pair 100 is located on the inside of each strut 130 as shown in FIG. 1A, that is, on the side facing the adjacent strut intersecting at the proximal peak 120. This minimizes the likelihood of adjacent struts 130 becoming hooked onto the barb pairs 100 during delivery when the anchor 110 is collapsed.

The proximal and distal facing barbs are preferably at least 2.0 millimeters (mm) in height off the strut 130. The proximal and distal facing barbs are preferably 2.0-4.0 mm in height off of the strut 130. They can, however, be 2.0-6.0 mm in height off of the strut 130.

In the embodiment of FIG. 1D, the proximally facing barbs 150 are at preferably at least 2.0 mm in height off the strut 130. The distal facing barbs 140 preferably protrude at least 2.5 mm in height off of the strut 130. If the barbs 100 are shortened below roughly 1.5 mm, the implant tends to migrate quickly.

The proximal facing barbs 150 are preferably short so as to make for easy removal of the device from the surrounding tissue. Removal and repositioning of gastrointestinal implants is described in U.S. application Ser. No. 11/318,083, filed on Dec. 22, 2005 and in U.S. application Ser. No. 12/005,049, filed on Dec. 20, 2007, herein incorporated by reference in their entirety.

The distal facing barbs 140 may be longer. If in the intestine, for example, even though peristalsis moves the implant in both directions, the overriding force is to pull the implant distal, thus the distal barbs 140 are longer to hold the device in place within the muscle. The distal facing barbs 140 may be up to 6.0 mm in height. Alternatively, the proximal and distal barbs may be an equivalent height off the strut 130.

The barb pairs 100 are made of 0.5 mm diameter, nitinol wire making them quite stiff. The barbs 100 are stiff enough to not be deflected by the soft tissues within the gastrointestinal tract. The range of diameters most effectively utilized in nitinol would be about 0.15-0.8 mm. Any diameter smaller than 0.15 mm results in a floppy barb that does not resist deflection well. Above a diameter of 0.8 mm, barbs 100 are so stiff that collapsing the anchor into a small tube for delivery becomes exceedingly difficult.

The barb pairs 100 are made of nitinol to facilitate elastic bending when they are loaded into the delivery capsule. Delivery capsules for gastrointestinal implants are described in U.S. application Ser. No. 11/057,861, herein incorporated by reference in its entirety. The angle of each barb of the pair 100 to the anchor strut 130 is preferably about 40 degrees. This angle could vary from about 20 degrees to about 90 degrees. Alternatively, the barb pairs 100 can be made of stainless steel. Alternatively, the barb pairs 100 can be coated with metal such as gold or platinum, or with plastics (such as polymers coated with anti-inflammatory drugs) to produce different (more or less) inflammatory responses.

Anchors having barb pairs 100 as shown in FIGS. 1A-1D, when used to anchor an implant in the intestine, have been shown to remain in position generally for 3-6 months, with elevated rates of migration after 6 months. In situations where longer term anchoring is desired, such as in the treatment of obesity or diabetes, controlled porosity of barbs can be used effectively. The barb with pores is inserted into tissue. This provokes a response from the tissue and causes the tissue to grow into the pores of the barb or otherwise interact with the pores, thus stabilizing its position within the gastrointestinal tract for a longer period of time as compared to using the non-porous barbs of FIGS. 1A-1D.

Preferably, the pores are open pores. This means that each pore or hole has a means of communication with at least one other pore, either within the barb or at the opposite end of a pore extending through the barb. This advantageously, allows living cells of the tissue to interconnect, thus further stabilizing the position of the anchor within the gastrointestinal tract. The barb preferably has an open space density from about 25% to about 75% to promote tissue ingrowth.

There are several techniques for adding porosity to the barb. One such technique is coating the barb with a porous substance. Another is to have pores within the barb itself or to cover the barb with a porous sheath. These techniques will be described below.

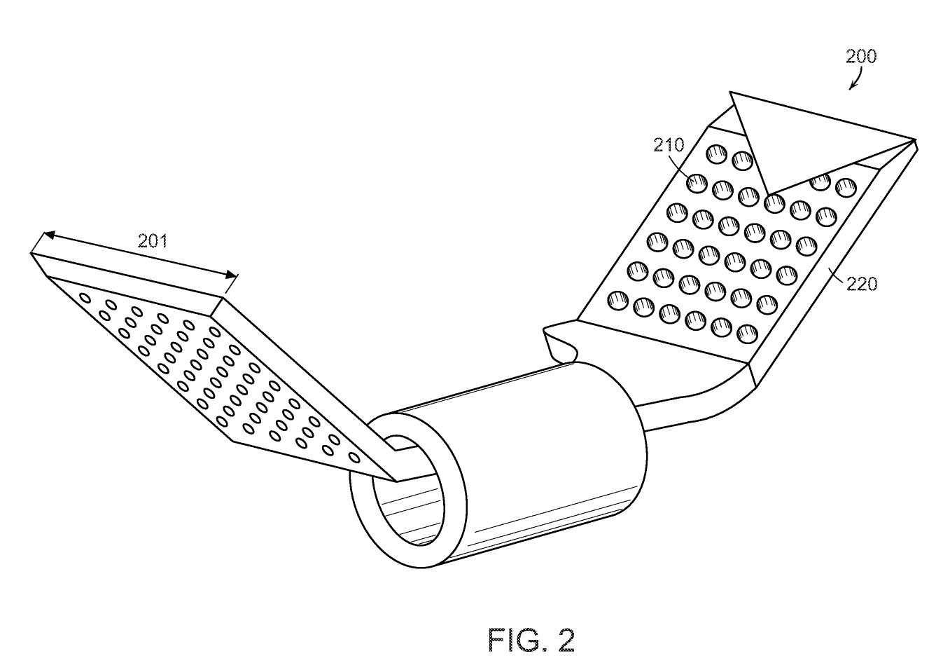

One embodiment of a paddle-shaped barb is shown in FIG. 2. Here the porous barb 200 is a paddle barb that is rectangular in shape and has pores 210. In this embodiment, the pores are drilled, mechanically or with a laser, or etched holes that extend entirely through a wall thickness 220 of the paddle barb 200. The pores can have a diameter of about 0.01-0.4 mm.

The paddle shaped barb 200 can have any suitable shape, such as circular, oval, rectangular, or any other suitable shape. A paddle barb 200, regardless of the shape, can preferably have a minimum thickness of 0.25-1.0 mm. Thus, the depth of the pore 210 is also 0.25-1.0 mm, or equivalent to the thickness of the barb 200.

The paddle barb 200 can have a width 201 of 1.0-2.0 mm. Advantageously, the wider surface area of the barb 200 provides a greater area in contact with tissue, and thus an increased retention force, while the thinness of the barb eases penetration into tissue. The paddle barb 200 can be flat, or can alternatively have a curved shape.

The paddle barb 200 is fixed to the anchor strut 130 similarly to the nonporous barb of FIG. 1. The proximal facing barb and distal facing barbs are preferably at a height of 2.0-4.0 mm above the anchor strut 130 as are the barbs of FIG. 1. The tip of the wider barb 200 can be shaped in various ways to either minimize or maximize tissue response to the barb.

Preferably, the tip of the barb 200 or any of the previous or subsequent barb embodiments is sufficiently sharp as to penetrate the gastrointestinal tissue. One example of sharpness testing is conducted using an Instron Tensile tester, the barb to be tested, and an adhesive backed polyester film with a thickness of about 0.1 mm. The film is mounted over a hollow cylinder in the bottom chuck of the tester. The barb to be tested in mounted in the upper jaw of the tester such that the point is perpendicular to the polyester film. The barb is then advanced slowly until the tip of the barb penetrates the film. The peak force is then recorded. The test results have shown that barbs that pierce easily with a peak force of less than 1 pound (lb), are unnecessarily sharp and may increase the risk of bleeding. Barbs that are excessively dull, having greater than 4 lbs of peak force, do not grip the tissue surfaces well enough to provide long term stability to the anchor. Thus, the barb preferably has a retention force of 1.25 to 3.0 lbs of penetration force.

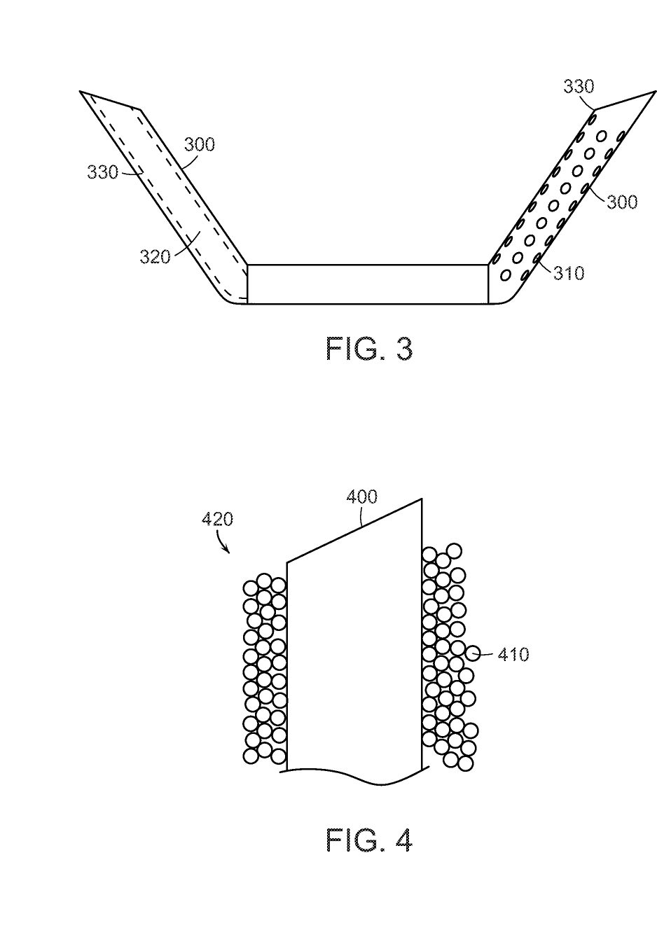

FIG. 3 shows an alternative embodiment of a porous barb. Here, the barb 300 is hollow with lumen 320. The barb 300 includes pores 310 in a barb wall 330. Each pore 310, is a hole that is drilled entirely through the barb wall 330. The pores 310 can have a diameter of 20-500 micrometers (μm). Preferably the pore diameter is 100-400 μm. Most preferably, the pores 310 have a diameter of 100-200 μm. All pores 310 can have an equivalent diameter or the pores 310 can have different diameters from each other.

The barb wall 330 preferably has a thickness of about 0.2 mm. Thus, each pore 310 also has a depth of about 0.2 mm. The barb preferably has a diameter (including hollow lumen 320) of about 0.25-1.0 mm. The inner diameter of the barb 300, is therefore the difference between the diameter of the barb 300 and the thickness of the barb wall 330. Advantageously, when the hollow barb 300 is inserted into tissue, tissue can grow in the pores 310 and also into lumen 320 of the hollow barb 300.

FIG. 4 shows an alternative and additional embodiment of a porous barb 400. Here the barb 400 is a sintered barb with sintered metal spheres 410 on the outside of the hollow or solid barb 400. The metal spheres 410 may be composed of any suitable metal material such as stainless steels or titanium.

The thickness of several communicating metal spheres 410 form a sintered coat 420. Each metal sphere 410 is about 25-100 μm in sphere size. The total thickness of the sintered coat 420, is thus about 100-300 μm or several metal spheres thick.

Only a portion of the barb 400 may be sintered with the metal spheres 410, for example, surrounding a barb substrate or only extending a partial length of the barb. Alternatively, the entire barb 400 may be of sintered metal spheres 410.

In one embodiment, to create the sintered barb 400, a glue may be applied to the barbs of FIG. 1. The barbs are then dipped in sieved, metal spheres 410 of the desired diameter. The spheres 410 may then be sintered onto the barbs using heat sufficient to weld the spheres 410 to each other and to the barbs 400. Alternatively or in addition, hollow barbs, or wide, flat barbs may also be treated in a similar way to create a sintered and porous surface.

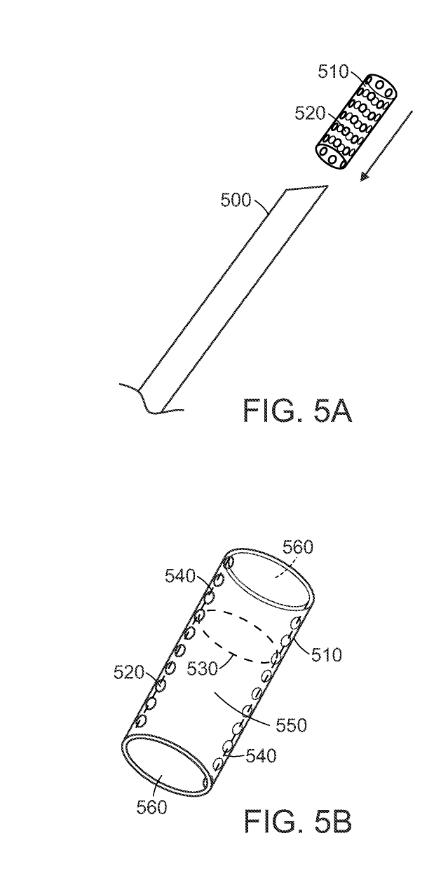

In another embodiment, the barb has a barb substrate 500 and a porous sheath or cover 510, as shown in FIG. 5A. The sheath 510 can be formed from a biocompatible plastic like silicone, pHEMA, urethane, or any suitable material. The sheath preferably has an open porous structure, meaning that pores 520 are in communication with each other. As shown in FIG. 5B, the sheath 510 has lumen 550 and an opening, 560 at each of its proximal and distal ends.

Each pore 520 can be 20-500 μm in diameter. Preferably, each pore 520 is between 20-100 μm in diameter. The sheath 510 has an open space density from 25% to 75%. The sheath 510 has an inner diameter 530 of 0.25-1.0 mm depending on the diameter of the barb on which it used. For example, if the barb is 0.5 mm in diameter, the sheath 510 should be made to tightly fit on the barb 500 substrate, with a diameter of about 0.25-0.4 mm. The sheath has a wall thickness 540 of at least 200 μm. The length of the sheath 510 can be from 2.0-4.0 mm depending on the length of the barb it is placed on. Alternatively, if the barb is a longer or shorter barb, the sheath 510 can be sized accordingly.

In operation, the sheath 510 is fitted onto at least a portion of the barb substrate 500. The tip of the barb substrate 500 can protrude out of an opening of the sheath 510. Alternatively, the tip of the barb substrate 500 may be completely within the sheath 510.

In operation in the intestine for example, the barb substrate 500 pierces through the muscle of the tissue in which the implant is being anchored. The muscle may then heal into the porous sheath material 510. As long as the muscle or other surrounding tissues are healed into the sheath 510, a bond is made between the tissue and the anchor, advantageously providing longer term stability. Additionally, with multiple barbs around the circumference of the anchor as shown in FIG. 1B, anchor stability is further improved.

Because the in-growth attachment of the tissue to the anchor may be at discreet points on the sheath 510, the implant device that is being anchored is preferably removable with minimal invasion to the tissue and surrounding anatomy. In one embodiment, this is accomplished by pulling on at least one drawstring located on the anchor to collapse the anchor, tearing each of the barb/sheath features out of the tissue. Alternatively, the barb may be directly pulled with an endoscopic grasper to remove it from the engaged tissue. The amount of trauma caused to the tissue is likely to be acceptable. In the event that the amount of trauma is unacceptable, the sheath 510 is designed such that the barb substrate 500 is separable from the sheath 510. In this way, as the anchor is collapsed and removed, the sheath 510 remains in the tissue.

Potential tissue damage can further be reduced by decreasing the amount of surface area involved in the healing between the tissue and the barb. The less surface area of tissue, the less the force required to remove the barb from the tissue and the less area of tissue traumatized.

In one embodiment (not shown), a barb substrate can have a textured surface to promote tissue in-growth. A texture can be added to the surface of the barb substrate by techniques such as roughening, scoring, grooving, threading, coating or any other suitable texturing mechanism. The texturing can preferably be added to the surface of a hollow barb of FIG. 3. The texturing can be added on discreet points of the barb or along the entire length of the barb. The texture can be added by providing pores on the surface of the substrate. In some embodiments, the pores can be closed pores, or pores that do not have a means of communication with other pores. Alternatively, or in addition, texturing can be added to the surface of any of the barbs shown in FIG. 1-5. The texture can be applied on discrete points of the barb or along the entire barb. Though texturing may promote tissue attachment, having pores such as those shown in FIGS. 2-6 is preferable and a more effective way of promoting ingrowth of tissue.

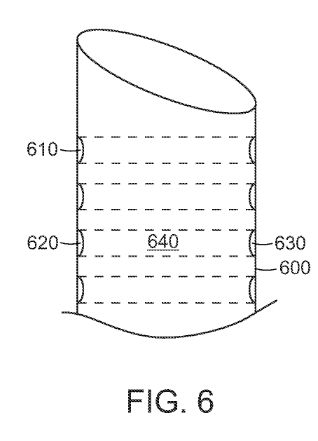

In an alternative embodiment, the rigid barb 600 has pores 610 as shown in FIG. 6. The pore 610 has a first opening 620, and a second opening 630 connected by lumen 630. Thus, the pore 610 extends all the way through a portion of the barb 600. This may be accomplished by laser drilling through the barb across its diameter. The depth of the pore can be from 5% to 50% of the diameter of the barb.

Advantageously, controlled porosity of the barbs increases the long term retention force of the implant within the gastrointestinal tract. Each of the porous barbs of FIGS. 2-6 has a retention force of 2.0-4.0 lbs as measured in animal tissues. This is substantially higher than a non-porous barb whose pullout force could not be measured as it was quite low. The increased retention force is advantageously a result of the porosity and thus, the increased stabilization due to tissue in-growth or other interaction.

As previously stated, the barbs are attached to an anchor used to anchor an implant or device in the gastrointestinal tract. Thus, an implant may be anchored in the esophageal region, the stomach, or the intestinal area. In a preferred embodiment however, the barbs are used with an anchor to anchor an implant for the treatment of obesity in the intestinal area. Gastrointestinal devices are described in U.S. Pat. No. 7,267,694, U.S. application Ser. No. 10/858,851, and allowed U.S. application Ser. No. 10/858,852, all herein incorporated by reference in their entirety.

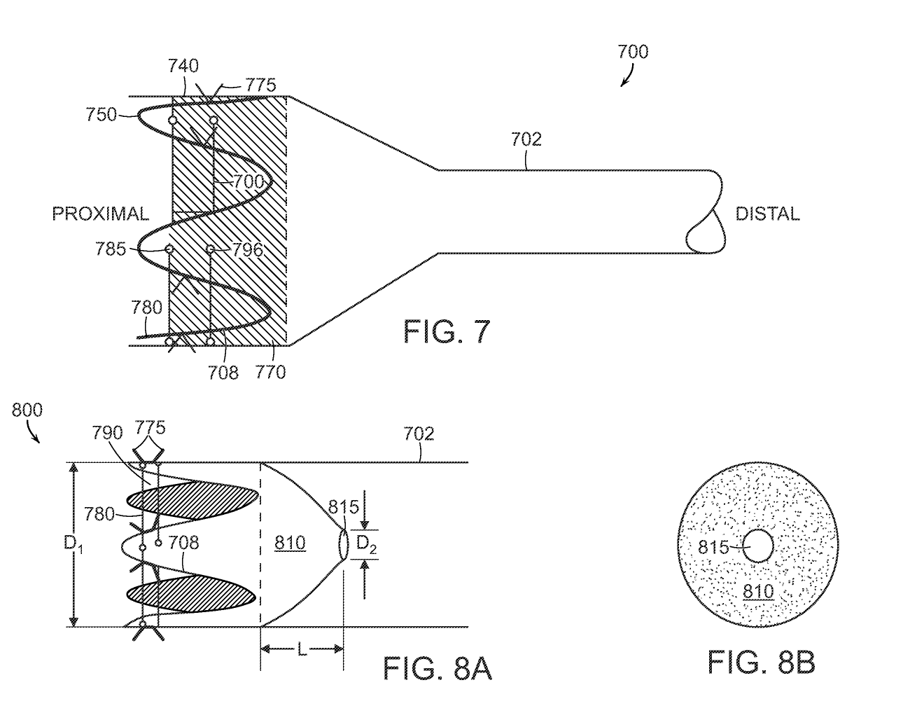

FIG. 7 is a side view of an exemplary gastrointestinal implant device 700. The gastrointestinal implant device 700 includes an elongated, open-ended, unsupported flexible sleeve or tube 702 having a first proximal opening and a second distal opening. Within the sleeve 702 is a passageway that extends from the first proximal opening to the second distal opening for transporting the chyme exiting the stomach. The surface of the passageway (the interior surface of the implant device 702) is smooth to enable the chyme to easily pass through. The exterior surface of the implant device 700 is smooth to be non-irritating to the bowel.

The sleeve material 702 is floppy, thin, and conformable so that it collapses in the intestine to a small volume to minimize bowel irritability. Also, the sleeve 702 has minimal hoop strength, so that it can fall flat until food passes through, thus minimizing interference with peristalsis. It has a low coefficient of friction (less than about 0.20) so that chyme slides easily through it and the bowel slides easily around it. Further, the low coefficient of friction prevents the sleeve from sticking to itself, thus making it easier for the sleeve 702 to open as chyme is pushed through it. It is of low or no permeability to fluids so that the chyme does not touch the bowel wall and the digestive enzymes do not breakdown the chyme. It is biologically inert and non-irritating to the tissues. One class of materials includes fluoropolymers. In some embodiments, the sleeve 702 is formed from expanded PTFE with a wall thickness of about 0.01-0.025 mm and an internodal distance of 20 μm. This material is hydrophobic but is slightly porous. However, these very small pores may plug over time. The porosity may be reduced by coating the material on the inside, outside or in the pores with dilute solutions of FEP, silicone or polyurethane.

Another material is polyethylene with a wall thickness of less than 0.001 inches. Other materials include Cast PolyTetraFluoroEthylene (PTFE), Cast PTFE with Fluorinated Ethylene Propylene (FEP), or Extruded FEP. These materials are solid and substantially non-porous in contrast to ePTFE which is porous, but these materials are also considered to be fluoropolymers. The wall thickness is preferably less than about 0.001 inches. Rubber-like materials typically have friction coefficients of about 1-4, significantly stickier than these materials. However, in alternate embodiments other materials having similar characteristics can be used.

In some embodiments, the sleeve 702 is formed using a combination of two or more materials. For example, the sleeve 702 can be formed using a combination of ePTFE and FEP. Such a combination can be formed by layering the two materials together and generally provides a low coefficient of friction while being substantially non-permeable. The ePTFE provides significant flexibility and softness while the FEP is used to seal the pores in the ePTFE making the material substantially non-porous. FEP is also thermoplastic permitting construction techniques using heat to fuse layers together. This material is used to form the sleeve as well as to cover both the outer and inner surfaces of the anchor.

The sleeve 702 includes two layers of material at least at the proximal end. A first outer layer covers the exterior of the anchor 708. The second inner layer covers the interior surface of the anchor 708. The barbs 775 protrude from the exterior surface of the anchor 708 through the first outer layer of the sleeve 702. The diameter of the sleeve 702 is selected such that the first outer layer of the sleeve 702 fits over the anchor 708.

The sleeve length is variable and can range from about one foot to about five feet. The typical length of the sleeve 702 is about 2 to 4 feet. The length of the sleeve 702 is selected to bypass the duodenum and a portion of the jejunum. The length can optionally be increased to further decrease absorption by bypassing a longer section of the jejunum. Thus, the length of the sleeve 702 is variable. The procedure is a less invasive alternative to surgery for the treatment of obesity and morbid obesity and also provides a new treatment approach for Type-2 diabetes.

Within the implant device 700 at the proximal end including the first proximal opening is a collapsible self-expanding anchor 708. The anchor 708 may be a collapsible self-expanding stent with struts. Alternatively, the anchor 708 may be a collapsible, self-expanding wave type anchor coupled to the proximal portion of the sleeve 702 as shown here. The wave anchor 708 includes adjacent interconnected struts 740 connected by wave peak 750. In one embodiment, the anchor 708 has ten struts.

The wave anchor 708 includes a compliant, radial spring shaped into an annular wave pattern, providing an outward radial force, while allowing substantial flexure about its perimeter. Such flexure is advantageous as it allows for minimally-invasive delivery and ensures that the device will substantially conform to the surrounding anatomical structure when implanted. The annular wave element can be formed from one or more elongated resilient members and defines a lumen along its central axis formed between two open ends. When implanted, the central axis of the anchor 708 is substantially aligned with the central axis of the duodenum, allowing chyme to pass through the device 700. Additionally, the compliant wave anchor 708 minimizes trauma to the tissue by providing sufficient flexibility and compliance, while minimizing the likelihood of tissue erosion and providing a solid anchoring point to the tissue.

The compliant wave anchor 708 can be manufactured from a resilient metal such as a heat-treated spring steel, stainless steel, or from an alloy such as NiTi alloy commonly referred to as Nitinol. Other alloys include nickel-cobalt-chromium-molybdenum alloys possessing a unique combination of ultrahigh tensile strength, such as MP35N. Additionally, the wave anchor 708 can be formed from a polymer and/or a composite having similar properties. The wave anchor 708 can be manufactured from a single strand, such as a wire, contoured into the desired shape. Alternatively, the wave anchor 708 can be manufactured from multi-strands of the same or different materials similarly contoured to the desired shape. In some embodiments, the wave anchor 708 can be cut into the wave shape from tubular stock of the desired material, such as Nitinol. The wave anchor 708 can be removably attached within the body using any of the methods described herein for securing an anchor 708, including the use of barbs 775 attached to, and/or formed on the anchor itself. Preferably, the anchor 708 is radially collapsible for endoscopic insertion.

The diameter of the anchor 708 is dependent on where the anchor is positioned within the gastrointestinal tract. The diameter of the duodenum is about 25.0-50.0 mm based on human anatomy variations. The anchor 708 is adapted to be retained within the duodenum, particularly in the duodenal bulb just distal to the pylorus. In one embodiment, the length of the anchor 708 is selected to reside within the bulbous duodenum. In the preferred embodiment, the length of the anchor is 32 mm while the relaxed diameter is from 50.0-55.0 mm. A device to be anchored in the esophagus would likely be 20.0-35.0 mm and in the stomach, 50.0-100.0 mm.

The intraluminal anchor 708 can also include at least a proximal drawstring 780 as previously described, to facilitate repositioning and/or removal. The drawstring 780 can be provided at a proximal end of the implant device 700 and be adapted for engagement by a removal device such as a hook. The drawstring 780, when engaged, can be pushed or pulled by the removal device, in opposition to the stationary intraluminal anchor, to at least partially collapse at least part of the intraluminal anchor. The anchor 708 can also include a distal drawstring 790 to further facilitate repositioning and/or removal. Gastrointestinal implants with drawstrings are described in U.S. application Ser. No. 12/005,049, filed on Dec. 20, 2007, herein incorporated by reference in its entirety.

The wave anchor 708 includes webbing material 770 between the struts 740 of the anchor 708. The webbing material 770 can be made of a class of materials including fluoropolymers and is preferably made of the same material as the sleeve 702. In some embodiments, the webbing material 770 is formed from expanded PTFE. Another material is polyethylene. Other materials include Cast PolyTetraFluoroEthylene (PTFE), Cast PTFE with Fluorinated Ethylene Propylene (FEP) or PerFluoroAlkoxy (PFA), Extruded FEP and Extruded PFA.

The anchor 708 includes the plurality of opposed barbs 775 for anchoring the implant device 700 to the muscular tissue of the duodenum. The barbs 775 include pairs of rigid, elongated barbs. Each side of the pair and are outwardly directed at fixed angles and in opposite axial directions. The barbs are of a length such that they are able to penetrate muscular tissue.

The barbs 775 can be any of the barbs of FIGS. 1-6. If shorter term anchoring is needed, the non-porous barbs of FIG. 1 should be used. If longer term anchoring is desired, any of the porous barbs of FIGS. 3-6 should be used due the increased stability as a result of tissue ingrowth.

In one embodiment, the gastrointestinal device may include an artificial stricture as shown in FIG. 8A. Gastrointestinal implants with artificial strictures are described in U.S. application Ser. No. 11/827,674, filed on Jul. 12, 2007, and in U.S. application Ser. No. 11/330,705, filed on Jan. 11, 2006, herein incorporated by reference in their entirety. An exemplary artificial stricture device 800 adapted for gastrointestinal applications is illustrated in FIG. 8A. The device 800 includes the anchor 708 coupled to an artificial stricture. The artificial stricture retards the flow of chyme therethrough. The anchor 708 is adapted to anchor the device 800 within the gastrointestinal tract. When placed at or below the pylorus, the stricture operates to slow gastric emptying. The anchor is adapted to hold the device securely in place under gastrointestinal forces and pressures.

The artificial stricture can be formed from a blocking material 810 coupled to the anchor 708, the blocking material defining the aperture 815 therein. The blocking material is dimensioned to at least cover the cross-sectional area of the lumen within which it is implanted. For an implant adapted for use in the proximal duodenum of an adult male, the diameter of the impermeable material would be at least about 25 millimeters.

The blocking material 810 may be constructed of a compliant or non-compliant polymer. If non-compliant, such as 0.0005 inches (0.013 mm) thick ePTFE and FEP, then the hole size remains fixed and also can be dilated with a balloon as it will plastically deform. If compliant, such as with 0.015 inches (0.38 mm) thick, low durometer silicone that is preferably below 30 A, the hole may enlarge in response to elevated pressures that result when the hole gets obstructed by large food particles. The blocking material 810 can alternatively or in addition be made of the same material described previously in the reference to intestinal sleeves.

The stricture is created by forming an aperture 815 having a reduced cross-sectional area, or diameter within the blocking material 810. The aperture can be formed, for example, by simply cutting or punching a hole of the appropriate dimensions into the blocking material 810. For example, the hole can be less than about 10 mm in diameter for an exemplary 25.0 mm implant. In some embodiments, the aperture is about 4.0 mm in diameter. It is unlikely, however, that an aperture of less than about 3.0 mm would be used in a human application as food particle passing through the pylorus are typically about 1.0-2.0 mm or less in size. The stricture is positioned distal to the pylorus and proximal to the ampulla of vater in the duodenal bulb.

In one embodiment, the aperture 815 is elastic and expandable under pressure from material flowing through the anchor 708 and the aperture 815 at elevated physiological pressures. The aperture 815 ranges in diameter from 1.0-10.0 mm over a range of water pressure. The aperture 815 expands substantially at a physiological pressure of 50 inches of water or more. The diameter of the aperture 815 is about 5.0 mm or below, and is preferably about 4.0 mm or below when the water pressure is between 0 inches and 50 inches of water. The diameter of the aperture 815 opens to greater than 4.0 mm when the water pressure is above 50 inches of water. The aperture 815 is made of a biocompatible material such as silicone.

As shown in FIG. 8A, the implant 800 can also include the flexible, floppy sleeve 702 that is at least one foot (0.3 meters) in length to extend into the intestine. The proximal end of the sleeve is attached to the anchor 708. The sleeve extends into the intestine and defines a lumen through which chyme passes. The implant further includes the restricting aperture 815 within the sleeve 702, wherein the aperture 815 is adapted to retard the outflow of food from the stomach.

Additionally, the anchor 708 includes one or more external barbs 775, further securing the implant device in the presence of peristalsis. Preferably, the barbs 775 are sized and positioned to engage muscular tissue. As previously stated, the barbs can be any of the barbs of FIGS. 1-6. If shorter term anchoring is desired, the barbs of FIG. 1 can be used. If longer term anchoring is desired, any of the porous barbs of FIGS. 2-6 can be used.

While this invention has been particularly shown and described with references to preferred embodiments thereof, it will be understood by those skilled in the art that various changes in form and details may be made therein without departing from the scope of the invention encompassed by the appended claims. It should also be appreciated that the various features of the embodiments that have been described may be combined in various ways to produce numerous additional embodiments. Moreover, while various materials, dimensions, shapes, implantation locations, types of barbs, etc. have been described for use with the disclosed embodiments, others besides those disclosed may be utilized without extending the scope of the invention, including implantation locations in or above the pylorus.

What is claimed is:

1. A gastrointestinal implant comprising:
an anchor; and
barbs attached to the anchor, the barbs having pores therein.

2. The device of claim 1, wherein the anchor is at least 50.0 mm in diameter.

3. The device of claim 1, wherein the gastrointestinal implant further comprises a floppy, flexible sleeve coupled to the anchor.

4. The device of claim 3, wherein the sleeve is at least a foot in length.

5. The device of claim 1, wherein the anchor is a wave anchor.

6. The device of claim 1, wherein the pore size of the barb is about 100-400 μm.

7. The device of claim 1, wherein the barb has an open space density between about 25% and 75%.

8. The device of claim 1, wherein the barb is 2.0-4.0 mm in height off of an anchor strut.

9. The device of claim 1, wherein the barb is a rectangular barb.

10. The device of claim 9, wherein the barb has a width of about 1.0-2.0 mm.

11. The device of claim 1, wherein the barb is a hollow barb.

12. The device of claim 11, wherein the barb has a diameter of about 0.5-1.0 mm.

13. The device of claim 1, wherein the barb is a sintered barb.

14. The device of claim 1, wherein the barb is a barb substrate having a porous sheath covering at least a portion of each barb.

15. The device of claim 14, wherein the porous sheath is made of a biocompatible plastic.

16. The device of claim 15, wherein the plastic is silicone.

17. The device of claim 14, wherein an inner diameter of the porous sheath is about 0.25-1.0 mm.

18. The device of claim 15, wherein the wall thickness of the porous sheath is at least 200 μm.

19. A method of attaching an implant comprising:
positioning an anchor of the implant within a natural bodily lumen;
piercing surrounding tissue with barbs that are coupled to the anchor, the barbs having pores therein; and
allowing tissue to interact with the pores in the barbs.

20. The method of claim 19, wherein the tissue grows completely into the pores.

21. The method of claim 19, wherein the barb is paddle shaped barb.

22. The method of claim 21 wherein the barb is a rectangular paddle shaped barb.

23. The method of claim 19, wherein the barb is a hollow barb.

24. The method of claim 19, wherein the barb is a sintered barb.

25. The method of claim 19, wherein the implant further comprises a floppy, flexible sleeve coupled to the anchor.

26. The method of claim 19, wherein the anchor is a wave anchor.

27. The method of claim 19, wherein the anchor is positioned at or distal to the pylorus.

28. The method of claim 19, wherein the barbs pierce muscle.

29. The method of claim 19, wherein the barbs further comprise a porous sheath covering at least a portion of each barb.

30. The method of claim 29, wherein the porous sheath is made of a biocompatible plastic.

31. The method of claim 30, wherein the plastic is silicone.

32. The method of claim 30, wherein an inner diameter of the porous sheath is about 0.25-1.0 mm.

33. The method of claim 29, wherein the wall thickness of the porous sheath is at least 200 μm.

34. A gastrointestinal implant comprising:
a flexible, floppy sleeve, open at both ends;
a self expanding anchor, and
barbs coupled to the anchor, each barb comprising a barb substrate, at least a portion of the barb substrate surface being textured with pores.
